(19) United States
(12) Patent Application Publication
(10) Pub. No.: US 2026/0041347 A1
(43) Pub. Date: Feb. 12, 2026

(54) DEVICES AND METHODS FOR MEASUREMENT OF EXCRETED MASS DURING URINATION

(71) Applicant: Unephra Inc., San Jose, CA (US)

(72) Inventors: Farhad TAGHIBAKHSH, San Jose, CA (US); Parastoo FAZELZADEH, San Jose, CA (US); Pedram FATEHI, Palo Alto, CA (US); Farhad BATMANGHELICH, San Jose, CA (US); Saba LAHSAEI, Lafayette, CA (US)

(73) Assignee: Unephra Inc., San Jose, CA (US)

(21) Appl. No.: 19/366,367

(22) Filed: Oct. 22, 2025

Related U.S. Application Data
(63) Continuation of application No. PCT/US2024/026090, filed on Apr. 24, 2024.
(60) Provisional application No. 63/497,966, filed on Apr. 24, 2023.

(51) Int. Cl. A61B 5/20; A61B 5/00; A61B 5/01

(52) U.S. Cl. CPC A61B 5/208; A61B 5/01; A61B 5/7267; A61B 2560/0223

(57) ABSTRACT

The present application discloses a urine monitoring device that can measure physiological parameters from a user's urine to assess certain cardiac, or cardiovascular, or other metabolic risks, or other disease risks, or the user's response to certain medications. The device is portable and can be handheld or mounted inside a toilet, or on a urinal. The device can authenticate the user, and upon authentication of a verified user collected data from the user's urine can be automatically transferred using secure data transfer protocols to the user's personal electronic devices such as a smart watch or a mobile phone, or to the user's other data accounts via the internet or cellular communication for further data processing, or for user's view, or for user's physician and care team to review. The device can also be used in conjunction with a urine catheter and urine collecting bags as used in hospitals to collect urine from hospitalized patients for a continuous monitoring of patients' key urine parameters, patients' response to certain medications, and patients' health in general during hospitalization.

### CROSS-REFERENCE TO RELATED APPLICATIONS

This application is a continuation of International Patent Application No. PCT/US2024/026090, filed on Apr. 24, 2025, which claims benefit of priority to U.S. Provisional Application No. 63/497,966 filed Apr. 24, 2023, which is incorporated herein by reference in its entirety.

### BACKGROUND

### Technical Field

The present disclosure relates to devices and methods for health monitoring and personalized medicine, in general, and collecting physiological information from urine for such measures in particular.

### SUMMARY

A portable urine monitoring device is disclosed that can be used for medical applications or personal health tracking. The device can have multiple sensors for measuring various physiological parameters of urine, such as, but not limited to, urine volume, excreted sodium, potassium, calcium, chloride, sulphate, and ammonium, specific gravity, pH, color and clarity, of any individual's urine who urinates onto the device; and said measurement can be performed during urination, and said device can be hand held, or mounted on a urinal, or on any regular toilet, or can be used in conjunction with a bladder catheter and urine collecting bags as used in hospitals; and said device is capable of measuring, recording, monitoring and processing said measured urine parameters to assess said individual's certain cardiac related risks, or certain cardiovascular related risks, or certain liver related risk, or kidney related risks, or said user's response to certain medications, and/or other physiological risks, and said device can transfer said measured urine parameters and/or risk assessment results to said individual's personal electronic device, or a cloud-based data processing and storage accounts, or other computer systems for further assessment, tracking, monitoring and/or other applications for personal viewing or professional review and monitoring by physicians via short range communication methods such as Bluetooth, or near field communication, or Wi-Fi, or via internet, or cellular communication, or by wire.

A system is disclosed to measure urine volume during urination by measuring the exchanged thermal energy between flowing urine at known temperature, with a body or bodies of known thermal capacity at known temperature, where the urine volume can be extracted from temporal change in temperature of one or more temperature sensors in contact with said bodies, over which the urine flows, using a pure physical model, or an enhanced physical model by machine learning and/or artificial intelligence, or purely using a machine learning and/or artificial intelligent algorithm.

A system is disclosed to measure urine volume during urination by measuring the impact of urine flow on mutual capacitance of multiple interdigitated electrodes beneath the surface over which the urine flows using a physical model enhanced by a machine learning and/or artificial intelligent algorithm.

A system is disclosed to measure urination time by examining temporal variation in temperature of one or more temperature sensors, over which the urine flows, and the urination time can be extracted from temperatures of said sensors using a pure physical model, or an enhanced physical model by machine learning and/or artificial intelligence, or purely from a machine learning and/or artificial intelligent algorithm.

A system is disclosed to measure urination time by examining temporal variation in electrical conductance of one or more liquid contact sensors, over which the urine flows, and the urination time can be extracted from signal of said sensors using a pure physical model, or an enhanced physical model by machine learning and/or artificial intelligence, or purely from a machine learning and/or artificial intelligent algorithm.

A system is disclosed to extract urination flow rate, and to evaluate urination manner in terms of continuality, by examining extracted urination volume from temperature sensors, and urination time from temperature sensors and/or contact sensors, using a pure physical model, or an enhanced physical model by machine learning and/or artificial intelligence, or purely from a machine learning and/or artificial intelligent algorithm.

A sensor is disclosed to measure ionic concentration of a specific chemical, such as, but not limited to, sodium, potassium, chloride, calcium, ammonium or hydrogen in a liquid medium such as urine, and said sensor is a four-terminal charge gated transistor, where the charge gate electrode can be coated by a charge absorbing layer exposed to the liquid medium, and the drain, source, and gate of said transistor can be set at a pre-determined voltage, and the drain-source current of said transistor can be modulated as the result of absorption of ions of said specific chemical on the said charge absorbing layer on the charge gate of said transistor, proportional to the concentration of said specific chemical in the liquid medium.

A system is disclosed to measure concentration of a specific chemical in urine using an array of four-terminal charge gated transistors, and in each transistor the charge gate terminal can be coated by an ion absorbing layer, and each transistor can have a different sensitivity to said chemical compared to other transistors in the array, and said concentration can be extracted from drain-source current of all transistors using a pure physical model, or an enhanced physical model by machine learning and/or artificial intelligence, or purely using a machine learning and/or artificial intelligent algorithm.

A system is disclosed to measure concentration of a specific chemical in urine using an array of ion-specific electrodes, and each electrode can have a different sensitivity to said chemical compared to other electrodes in the array, by employing a different electrode geometry and/or different ion-specific membrane, and said concentration can be extracted from all electrode signals using a pure physical model, or an enhanced physical model by machine learning and/or artificial intelligence, or purely using a machine learning and/or artificial intelligent algorithm without using a reference electrode.

A system is disclosed to measure concentration of multiple chemicals in urine using an array of four-terminal charge gated transistors, and in each transistor the charge gate terminal can be coated by an ion absorbing layer, and each transistor can have a different sensitivity to said chemicals compared to other transistors in the array, and said concentration of chemicals can be extracted from drain-source current of said transistors using a pure physical model, or an enhanced physical model by machine learning and/or artificial intelligence, or purely using a machine learning and/or artificial intelligent algorithm.

A measurement system is disclosed using which, the specific gravity of urine, or concentration of certain chemical compounds in the urine can be extracted from analyzing signals from a multitude of electro-chemical sensors, ion specific electrodes, or charge gated transistors, spectrometry sensors and alike, using a physical model, or an enhanced physical model by machine learning and/or artificial intelligence, or purely a machine learning and/or artificial intelligent algorithm, where the number of measured parameters is smaller than the number of sensor signals used.

A system is disclosed for measuring excreted amount of certain chemicals in urine such as, but not limited to, sodium, sodium chloride, potassium, potassium chloride, in which the concentration of said certain chemical in the urine can be extracted using arrays of electrochemical sensors such as four-terminal charge gated transistors, and urine volume can be extracted from temperature sensors, and excreted amount of said certain chemical is calculated by the product of said concentration by said urine volume.

A system is disclosed that can be activated by any user to start the urine test device described above, wirelessly using said user's smart device, or by pressing buttons on a key pad, or by user's voice, and said user can be authenticated by the user's smart device, or the key combination, or a voice recognition mechanism, such that the system can perform a urine test and assigns the test results to said authenticated user.

A system is disclosed that can measure urine volume using temperature sensors as described above when a user urinates on said system and said system can collect all or part of the urine volume and conduct said collected urine via one or multiple passages to the location of multiple sensors for proper measurement of certain urine parameters as described above.

A system is disclosed that can automatically apply one or multiple calibration 24 solutions to the multitude of sensors described in above urine test systems embodiments for calibration of the sensors and can adjust the gate or source or drain voltage of the charge gated transistors such that said charge gated transistor sensors are always at best calibration.

### BRIEF DESCRIPTION OF THE DRAWINGS

FIG. 1A illustrates an example of a system diagram for measuring urine volume using temperature sensors, and an example characteristics of temperature sensors used.

FIG. 1B illustrates an example of a system diagram for measuring urine volume using capacitive sensors, and an example of processed sensors signals.

FIG. 2 illustrates a cross-section view of a four-terminal charge gated transistor and its key layers employed as an ion-specific sensor. The configuration of the charge gate transistor is side-gate.

FIG. 3 illustrates two other examples of ion-specific charge gated transistor a bottom gate version (300), and a top gate (305) version.

FIG. 4 illustrates one other variation of a top-gate charge gated transistor employed as ion-specific sensor.

FIG. 5 illustrates an example of a system for measuring concentration of a specific chemical in a liquid medium based on an array of ion-specific sensors.

FIG. 6 illustrates an example of a system for measuring concentration of multiple chemicals in a liquid medium based on an array of ion-specific sensors.

FIG. 7 illustrates an example of system components for measuring various urine parameters using the measurement system connected to a catheter and urine collecting bag.

FIG. 8 illustrates another example of system components for measuring various urine parameters using the measurement system connected to a catheter and urine collecting bag.

FIG. 9 illustrates an example of the portable urine test system and its most likely places to use.

FIG. 10 illustrates an example for the inside of the portable urine tester system with some details on internal urine passage and location of multiple sensors used.

FIG. 11 illustrates an example for the system diagram of various functional blocks of the portable urine tester.

FIG. 12 illustrates an example of the overall connectivity for the portable urine tester.

FIGS. 13A through 13E show various exemplary measurement data graphs for testing of a variation of the measurement system.

### DETAILED DESCRIPTION

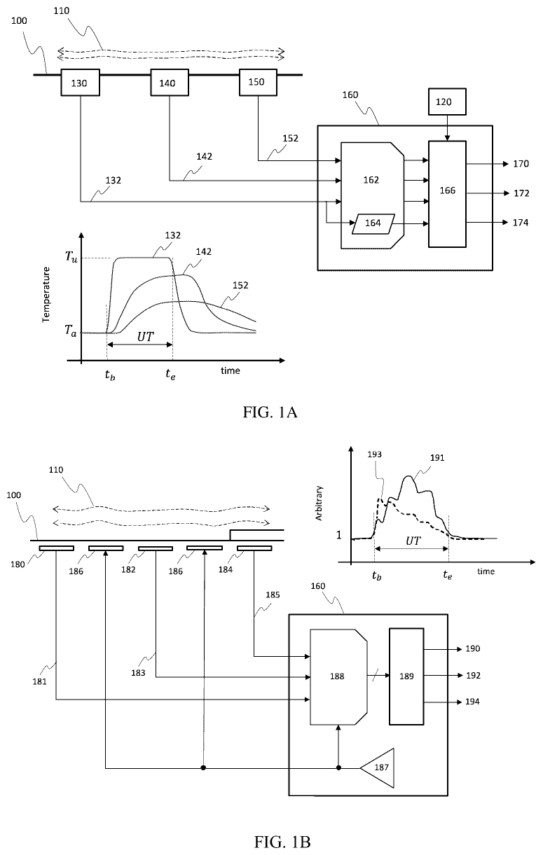

FIG. 1A illustrates a system for measuring urine volume using temperature sensors that has a body 100, where urine 110 can be poured over, or can flow over it such that it contacts with multiple temperature sensors 130, 140 or 150, which are also thermally in contact with the environment, and are built such that the maximum temperature they can measure can be written as:

$${T}_{MAX} = a {T}_{U} + ( 1 - a ) {T}_{E} \qquad ( 1 )$$

where α varies between 0 & 1, and there is at least one temperature sensor with α=1, for example sensor 130 measuring urine temperature T_{U}, and one sensor with α=0, for example sensor 120 measuring environment temperature T_{E}, and one sensor with 0<α<1 such as sensors 140 or 150. Additionally, sensor 130 has the fastest response time, as its corresponding signal 132 reaches T_{U} in the shortest time as shown the temporal graph, and the sensor 140 has a slower response time indicated by the slope of the signal 142, and sensor 150 has the slowest response time as indicated by the example response 152. One way to realize this is to expose the temperature sensor to both the urine flow and to the environment at different degrees. That means, designing the sensor in such a way that said temperature sensor has different thermal resistance to the urine flow and to the environment. For example, if a temperature sensor is directly exposed to the urine flow, but is thermally isolated from the environment, then a would be close to unity. On the other hand, if a temperature sensor is thermally connected to the urine flow and to the environment through similar thermal resistances, then a would be close to one half (0.5). Additionally, temperature sensors can be connected to a constant temperature heatsink using thermo-electric coolers (TECs) as opposed to be just coupled to the environment. This way, T_{E} is replaced by T_{C} which is the cold side temperature of the TEC. Temperature sensors can be analog or digital, and 160 processes sensors signals. If sensors are analog their signal can be converted to their digital representative signal by an analog-to-digital converter (ADC). Sub-system 162 can sample sensors' signals at a particular time within UT or outside of it, and the signal from the fastest responding sensor, i.e., 132 can be used to extract start time t_{b} and end time tₑ by the edge detector 164 to arrive at urination time UT by taking the difference. Sampled signals are further processed by sub-system 166 to generate urine volume 170, urination time 172, and the flow rate 174. Data processing within can be done using a pure physical model. For example, the amount of heat q exchanged at any time from urine flow at the rate of ϕ with sensor 140 can be written as q=γ(T_{U}−T_{E}), where the proportionality factor γ is the heat transfer coefficient depending on design of the sensor 140 and its interface with the body 100. Integrating this over urination time UT will result in:

$${\int }_{{t}_{b}}^{{t}_{e}} q \cdot dT = {\int }_{{t}_{b}}^{{t}_{e}} γ ( {T}_{U} - {T}_{E} ) φ \cdot dT \to {Q}_{T} = γ ( {T}_{U} - {T}_{E} ) {V}_{U} \qquad ( 2 )$$

in which Q_{T} is the total heat transferred from urine to 140, and V_{U} is the urine volume. With a finite heat energy transferred to sensor 140, its maximum temperature over T_{E} can be written as:

$${T}_{142 - max} - {T}_{E} = \frac{{Q}_{T}}{{C}_{d}} = \frac{γ}{{C}_{d}} ( {T}_{U} - {T}_{E} ) {V}_{U} \qquad ( 3 )$$

Therefore, the urine volume can be written to be proportional to the maximum temperature difference between sensor 140 and the environment temperature:

$${V}_{U} = \frac{{C}_{d}}{γ} \frac{{τ}_{140 - {max}^{- {T}_{E}}}}{{T}_{U} - {T}_{E}} \qquad ( 4 )$$

Where dynamic heat capacity C_{d} depends on material and design of the sensor 140, its interface with the body 100. The factor

$$\frac{{C}_{d}}{γ}$$

is different for each sensor and can be measured by experiment as C₁₄₂ for sensor 140. Therefore, the urine volume measured from sensor 140 can be written as:

$${V}_{U} = {C}_{140} \frac{{τ}_{142 - {max}^{- {T}_{E}}}}{{T}_{U} - {T}_{E}} \qquad ( 5 )$$

The dynamic range is limited where linear relation is established between sensor's maximum temperature and urine volume. Other sensors such as 150 can be designed to have different proportionality factor C, such that, C₁₅₀>C₁₄₀, to extend the dynamic range beyond that of sensor 140. In this case 166 can decide which sensor or sensors to incorporate for calculating urine volume. This can be done by comparing sensor measured temperature with the maximum possible temperature T_{MAX} which basically depends on design factor a and environment temperature T_{E}. For example, linearity can be assumed when sensor measured maximum temperature is within 15% and 85% of temperature span from T_{E} to T_{MAX}:

$${T}_{E} + 15 \% ( {T}_{MAX} - {T}_{E} ) \leq {T}_{sensor - max} \leq {T}_{MAX} - 15 \% ( {T}_{MAX} - {T}_{E} ) \qquad ( 6 )$$

With this, urine volume can be written as a linear function of multiple sensors measured temperatures as:

$${V}_{U} = \frac{1}{{T}_{U} - {T}_{E}} {\sum }_{i} {b}_{i} {C}_{i} ( {T}_{i - max} - {T}_{E} ) \qquad ( 7 )$$

For all sensors used, and coefficients b; are intelligently chosen to be non-zero for sensors that their maximum measured temperature is within the 15% to 85% of the temperature span from T_{E} to T_{MAX} as stated above, or zero otherwise. Coefficients Cᵢ can be derived from calibration experiment by minimizing the error between number of actual urine volume samples used in the calibration experiment and calculated value based on the above.

Alternatively, the sub-system 166 can be configured to implement an enhanced physical model by machine learning algorithm. For example, like equation (5), urine volume can also be extracted from temperature difference between sensor max temperature and Urine temperature, written as:

$${V}_{U} = {C}_{140}^{′} \frac{{T}_{U} - {T}_{E}}{{T}_{U} - {T}_{142 - max}} \qquad ( 8 )$$

And the two equations (5) & (8) can be combined to enhance linearity and extend dynamic range such that urine volume can be written as:

$${V}_{U} = {C}_{140}^{″} \frac{{τ}_{140 - {max}^{- {T}_{E}}}}{{T}_{U} - {T}_{140 - max}} \qquad ( 9 )$$

Therefore, for an array of sensors the above equation (9) can be written as:

$${V}_{U} = {\sum }_{i} {b}_{i} {C}_{i}^{″} \frac{( {T}_{i - max} - {T}_{E} )}{( {T}_{U} - {T}_{i - max} )} \qquad ( 10 )$$

Where coefficients C″ᵢ can be calibrated by minimizing error between calculated volume and actual volumes of a training set, i.e., urine samples with known volume, known temperature and known flowrates applied to the system, or by using other machine learning methods with said training set. Alternatively, in the above equation, the maximum sensor temperature Tᵢ₋ₘₐₓ can be replaced by the value of sensor at tₑ, i.e., Tᵢ(tₑ). In this case, different values for coefficients

$${C}_{i}^{″}$$

will be obtained compared to when Tᵢ₋ₘₐₓ is used.

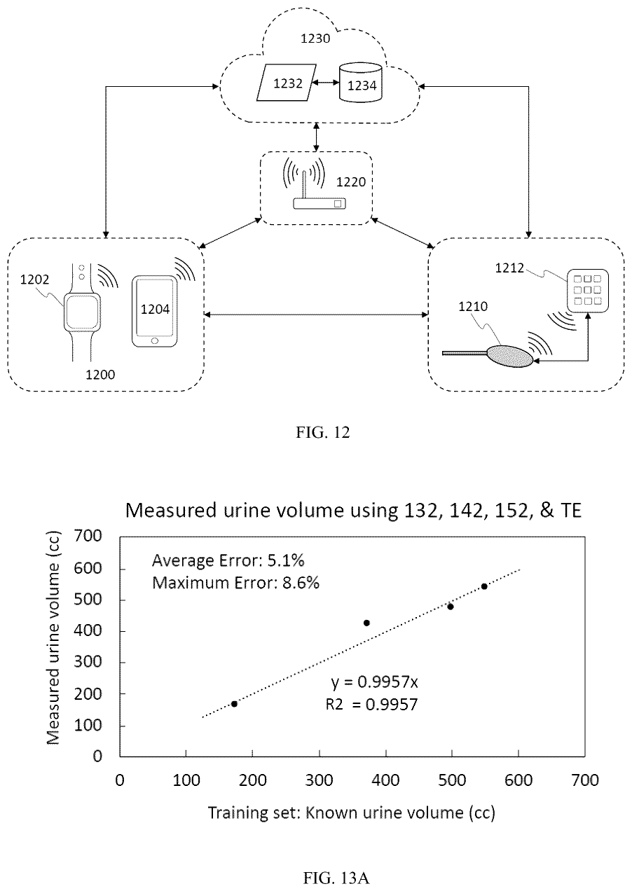

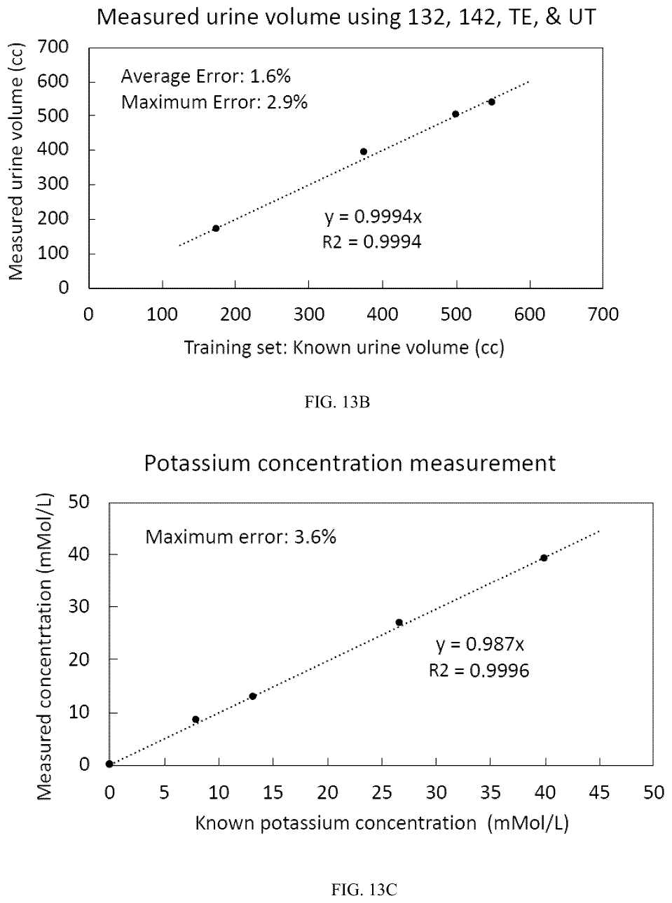

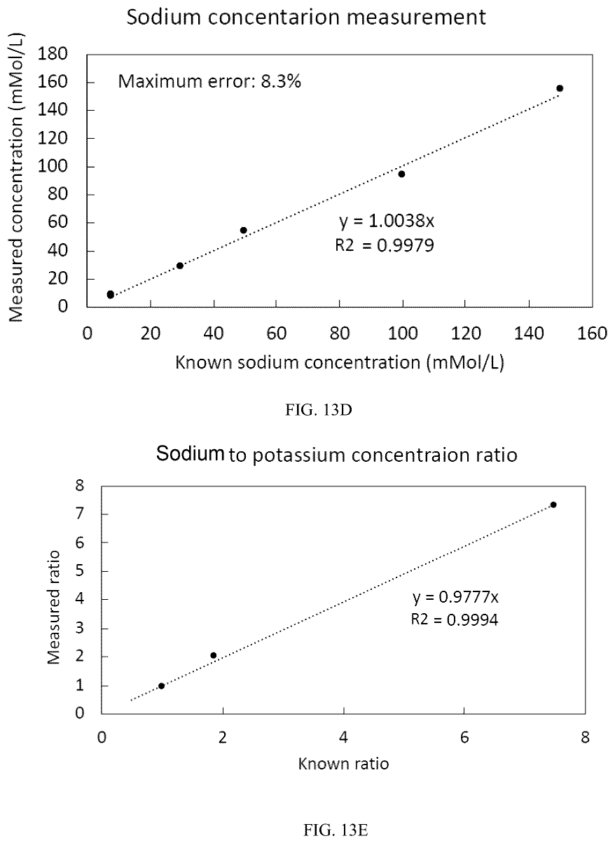

An example of measured urine volume using equation (10) from real urine samples of different volume and flow rates applied to temperature sensors of FIG. 1 is provided in FIG. 13A, where

$${C}_{i}^{″}$$

coefficients were optimized using linear regression.

Other intelligent optimizations are also possible on the fly. For example, when some sensors' signals are low and have noise, they can be dropped out from volume calculation equation and other parameters, not necessarily temperature values, incorporated in. For example, data from the same training set above can be processed by removing sensor signal 152 and adding urination time UT in the equation (10). The result in FIG. 13B shows much lower error and better linearity.

In addition to calculating urine volume 170, sub-system 166 can be further configured to calculate urination time 172, and flow rate 174.

FIG. 1B illustrates an example of the system for measuring urine volume using multiple capacitive sensors coupled to the surface 100 where the urine 110 flows over. The capacitive sensor is built using inter-digitized electrodes distributes under the surface 100, with three groups of sense electrodes, such as sense electrodes 180 and 182 that urine flows over them, and reference electrodes such as 184 that are kept away from urine flow, and drive electrodes such as 186 that are driven by signal generator 187. Sense electrodes can be one, to, or more, and coupling capacitance between the surface 100 and each sense electrode is not necessarily the same. Subsystem 188 process electrode signals by normalizing signals 181 and 182 by the reference signal 184 and integrating the envelope of normalized signal over the urination time UT as described below.

In FIG. 1B, signal 191 represents the envelope of signal 181 normalized by peak value of the reference signal 185, and similarly, signal 193 represents the envelope of signal 183 normalized by the peak value of reference signal 185. For each of the sense electrodes the area under the curve of the normalized signal is calculated from as follow, where ENV₁₈₁ is the envelope of signal 181, and |Ref₁₈₅| is the peak value of the reference signal.

$${A}_{181} = {\int }_{{t}_{b}}^{{t}_{e}} ( \frac{{ENV}_{181}}{\vert {Ref}_{185} \vert} - 1 ) {d}_{t} \qquad ( 11 )$$

Sub-system 189 performs ML estimation from processed signals generated by 188 such as area under the curve Aᵢ values to calculate urine volume 190, urination time 191, and flow rate 192 as described below.

Urine volume V_{U} can be written as a linear function of area under the curve for each sense signal as follow:

$${V}_{U} = {\sum }_{i} {c}_{i} {A}_{i} \qquad ( 12 )$$

Where coefficients cᵢ can be calibrated by minimizing error between calculated volume and actual volumes of a training set for a given sensor design.

Sub-system 198 also calculates urination time UT from thresholding the envelop signals to fine begin time t_{b} and end time tₑ, and calculating the difference as UT. Urine flow rate 192 is calculated from the ratio of urine volume V_{U}, over urination time UT.

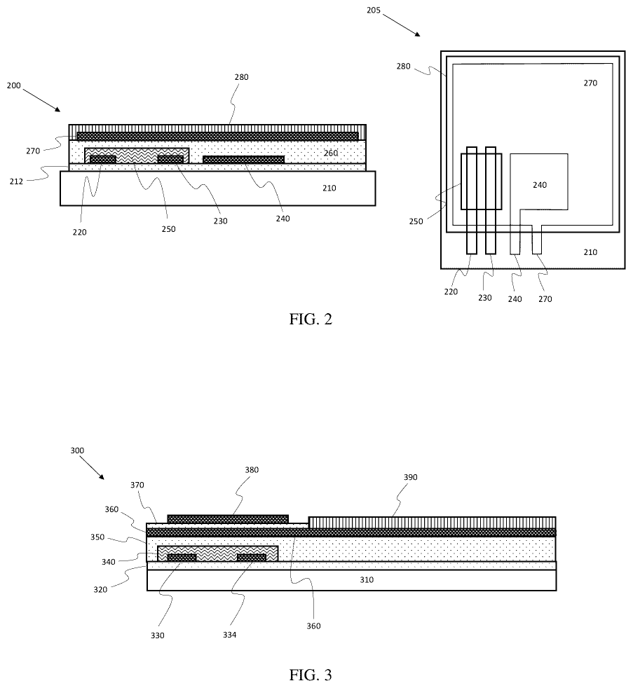

FIG. 2 illustrates an example of a novel solid state four-terminal ion-specific charge gated transistor sensor with calibration control that can be used to measure concentration of a specific chemical in liquid media, for example, concentration of potassium ions in urine. the device has four terminals: a drain (220), a source (230), a gate (240), and a charge gate terminal (270). The device with cross-section 200 can be made on a substrate 210 which can be a glass plate, or built into a silicon wafer, or alike, and said substrate can be coated by a buffer layer 212, upon which three electrodes, a drain (220), a source (230) and a gate (240) can be formed. Said drain and source are connected by the semiconductor layer 250 which can be from amorphous silicon, poly silicon, crystalline silicon or any other semiconductor material such as metal oxide semiconductors and their alloys. The dielectric layer 260 separates the charge gate electrode 270 from the gate electrode 240, and the semiconductor layer 250, and an ion-absorbing layer 280 on top of the charge gate electrode, which contacts the liquid media where various chemical ions are present. Layer 280 can comprise of different materials. For example, an ion-specific polymer membrane on top of an inorganic ion-trapping silicon compound layer such as silicon nitride, or silicon oxide, or amorphous silicon layer. The material of the absorbing layer can be chosen such that it has higher preference in absorbing ions of a certain chemical more than other ions. The electrical characteristics and operation of a charge gated transistor device has been fully explained in [U.S. Pat. No. 8,199,236-B2, US-20090147118-A1, and U.S. Pat. No. 7,995,113-B2]. Here, the number of absorbed ions on the ion-absorbing layer 280 depends on the concentration of the said ions in the liquid, and it results in change of threshold voltage of the transistor. Therefore, under a specific bias, the drain-source current of the charge gated transistor is modulated by the concentration of the ions in the liquid medium without the need for a reference electrode in the liquid. Below equation (11) explains the dependency of the drain-source current I_{DS} to the adsorbed ions/charges Q_{S} when the transistor is biased in the linear region. The absorbed charge Q_{S} changes the intrinsic threshold voltage V_{T} by

$$\frac{{Q}_{S}}{{C}_{Scq}} ,$$

where C_{cg} is the total capacitance of the charge gate. While the bias applied to the drain-source, i.e., V_{DS} acts as the gain of the sensor, the bias applied to the gate-source electrodes acts as the offset, and both V_{DS} and V_{GS} bias voltages can be used to adjust device calibration as it may drift over the course of the sensor life.

$${I}_{DS} = K ( {V}_{GS} - {V}_{T 0} - \frac{{Q}_{s}}{{C}_{c q}} ) {V}_{DS} \qquad ( 13 )$$

The performance of the charge gate transistor depends on the efficiency of the charge gate to absorb ions and converting the collected charge into a voltage that affect the threshold voltage of the transistor. This efficiency is determined by the value and ratio of various coupling capacitances among different electrodes of the device. The top view of the ion-specific charge gated transistor sensor 205 shows that the charge gate 270 can be extended independent the semiconductor 205, or the gate 240 allowing to optimize the capacitance values and ratios independently to arrive at an optimum characteristics and performance. Additionally, formation of the drain, source, and the gate electrodes under the charge gate maximizes the sensor aperture, i.e., the ratio of the charge gate area to the total device area, allowing to maximize sensitivity per area.

FIG. 3. Illustrates an alternative design of the ion-specific charge gated transistor sensor shown in FIG. 2, where the drain 330, source 334, semiconductor channel 340, and gate 380 are not formed under the charge gate 390 but are patterned on the side of it. This allows independent thickness control for dielectric layers 350 & 370, while in FIG. 2, it is not possible to make thickness of dielectric layer between gate 240 and charge gate 270 smaller than the dielectric thickness between charge gate 270 and semiconductor channel 250. Independent control of dielectric layers thickness provides more degrees of freedom for optimizing device capacitances for an improved performance. Additionally, the device 300 may provide better insulation between ion absorbing layer 390 and the semiconductor channel 340 & gate dielectric 350 to prevent migration of alkaline ions that may present in the liquid in contact with 390.

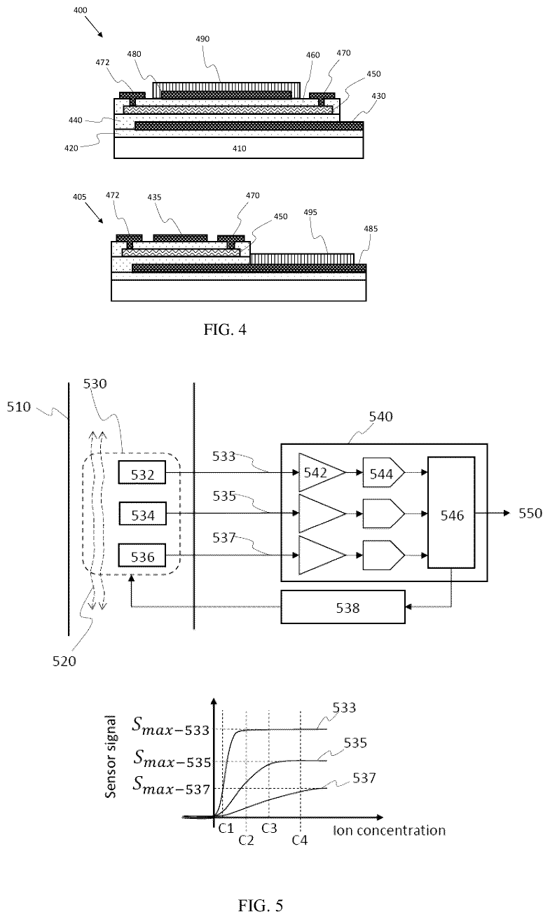

Different from FIG. 2 and FIG. 3, other structural configurations for the ion-specific charge gate transistor sensor are also possible. Two examples are shown in FIG. 4 where drain 470 and source 472 are formed on top of the semiconductor layer 450, and the two gates of the transistor are on opposite sides of the semiconductor layer. In device 400, the gate 430 is beneath the semiconductor layer 450, and the charge gate layer 480 is on top. While in device 405 the charge gate 485 is beneath the semiconductor channel 450, and gate 435 is on top. In these devices the charge gate does not affect the threshold voltage of the gate, but it acts as a second gate for the semiconductor layer, and they can be made to have the least coupling capacitance between the gate and the charge gate. Such devices are suitable for sensing in liquids with where ionic concentrations are high. The operation of device illustrated in FIG. 4 could be different from devices shown in FIG. 2 and FIG. 3. One way to operate these sensors is to initially charge the charge gate such that it puts the semiconductor layer in sub-threshold region. Then apply a pulse to the gate to drive the device in s-b threshold regime or switch it completely OFF.

Additionally, device 405 may provide better isolation of the semiconductor later 450 and gate dielectric 460 from diffusion of alkaline ions present in the liquid in contact with the ion-absorbing layer 495.

FIG. 5 illustrates an example of a chemical measurement system based on an array of ion-specific charge gated transistor sensors and method of processing their signals for enhancing accuracy and widening dynamic range of extracting concentration of a specific chemical ion such as potassium in a liquid medium such as urine. The sensor array 530 is mounted on a body 510 that can be a pipe, or any surface that exposes the sensor array 530 to the urine flow 520. The sensor array 530 can contain several individual sensor elements such as 532, 534, and 536 which can be ion-specific charge gated transistor sensors. The number of sensor elements on the sensor array can be two or more. The sensor elements are biased by the bias sub-system 538 that can provide required bias voltage for the gate, drain and source of the sensor elements, and the pre-set charge for charge gates. Additionally, 538 is responsible for adjusting calibration of individual sensor elements based on the time and their exposure to the chemical under measurement.

Each sensor element is configured or manufactured in such a way that provides different sensitivity and dynamic range. For example, the sensitivity of sensor 532 is more than that of sensor 534, and sensitivity of sensor 534 is more than that of sensor 536 as shown in the graph of sensor signal versus ion concentration, and as shown in the example graph, the dynamic range of sensor 536 is larger than that of sensor 534, and dynamic range of sensor 534 is larger than that of sensor 532. This allows the measurement system operates with better accuracy over a wide dynamic range compared to when a single sensor is used. The processing of sensor array signals to arrive at the ionic concentration 550 can be done using 540 which is comprised of several sub-systems. Firstly, analog signals from sensors, for example sensor signal 533, is conditioned and amplified by 542, and is converted to digital and sampled by 544. Each sensor's signal goes through the same chain of signal processing, either by having a designated channel for each sensor element, or multiplexing sensor's signals to a single signal processing channel. Sub-system 546 can process the digitized signals and calculate the concentration of desired chemical ion 550. One example of such processing is by establishing a linear function between the concentration of the ionic chemical, CX and sensors' signals as:

$$CX = {\sum }_{i} {C}_{i} ( {S}_{i} ) \cdot {S}_{i} \qquad ( 14 )$$

Basically, for all the sensors used, the offset corrected response of the sensor is calculated as Si, and then a corresponding coefficient Cᵢ is intelligently chosen based on the sensor's repones Sᵢ. For example, in the sensor response graph of FIG. 5, if CX is around C₁ then only sensor response 533 is used with a non-zero Cᵢ for 533, and Cᵢ will be set to zero for 535 and 537 because they are too close to zero and noise would be high for those low-level signals. If CX is around C₂, Cᵢ will be set to zero for 533 because it is outside of linear region and too close to saturation, and only 535 and 537 will be used for calculating CX with non-zero Cᵢ. Similarly, id cx is around C₃ to C₄, only 537 will be used for calculating CX and Cᵢ will be set to zero for 533 and 535 because both signals are at or close to saturation. This processing is performed by sub-system 546, and the corresponding coefficients Cᵢ can be extracted by minimizing error between calculated CX and known concentrations of the ionic chemical of interest in a base liquid medium applied to the sensor array 530.

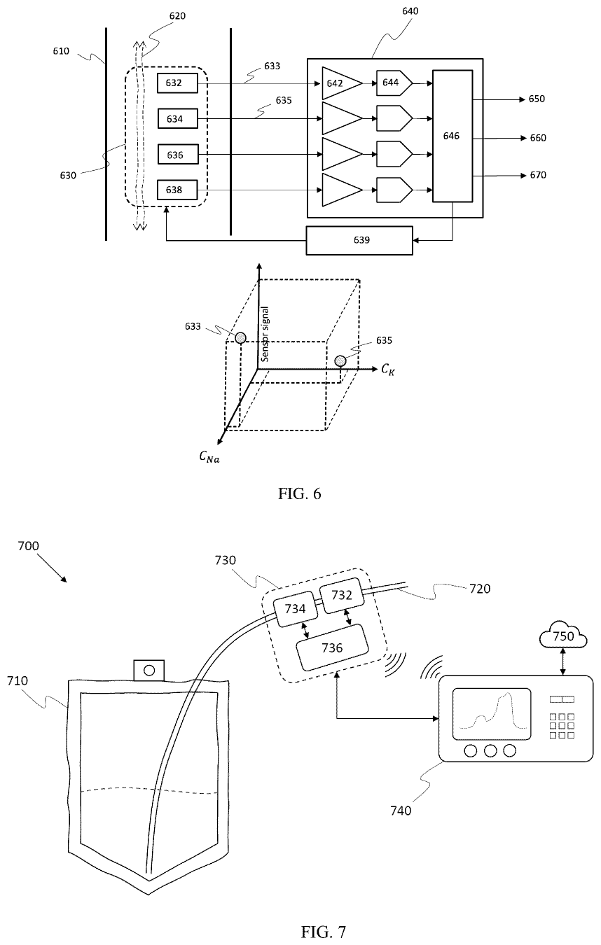

FIG. 6 illustrates an example of a chemical measurement system based on an array of ion-specific charge gated transistor sensors and method of processing their signals that can be used to improve selectivity, enhance accuracy and increase dynamic range of extracting concentration of two or more specific chemical such as potassium, sodium and hydrogen (pH) or alike from the same in a liquid medium such as urine. The working of the sensor array system is similar to what explained for the system illustrated in FIG. 5, only the design for individual sensor elements in the array 630, and the data processing 640 and calibration can be different.

Although the ion-specific absorber coatings used in charge gated transistor sensors or ion-specific membranes used in ion-specific electrodes could have high selectivity for a particular ion, but they also show non-zero sensitivity to a number of other chemical ions which makes selective and accurate measurement of the chemicals quite challenging. The system in FIG. 6 can help improve selectivity and accuracy of such chemical measurements. For example, if measurement of Sodium and Potassium concentration is intended, sensor response for an individual sensor element of the array can be written as:

$$S = a {C}_{Na} + {bC}_{K} \qquad ( 15 )$$

Where C_{Na} and C_{K} are the concentration of Sodium and potassium in urine, and a and b are sensor's sensitivity to sodium and Potassium respectively. The sensor array can be designed by choosing various combinations of a & b for array elements. For example, (high a, low b), (moderate a, low b), (low a, moderate b), and (low a, high b) for a four-element sensor array. For example, in the C_{Na}-C_{K} plane illustrated in FIG. 6, sensor response 633 represents (high a, low b), and 635 represents (moderate a, low b) sensor response. After amplification 642, digitization and sampling by 644, all sensors' signals can be processed by 646 to extract sodium concentration 650, and potassium concentration 660 using same sensors based on below equations:

$${C}_{Na} = {\sum }_{i} {C}_{i}^{Na} ( {S}_{i} ) \cdot {S}_{i} \qquad ( 16 )$$

$${C}_{K} = {\sum }_{i} {C}_{i}^{K} ( {S}_{i} ) \cdot {S}_{i} \qquad ( 17 )$$

As indicated by above equations, same sensors' signals can be used for extracting sodium and potassium concentration (or any other ionic chemical) using proper set of coefficients for the linear function. These coefficients, for example,

$${C}_{i}^{Na} and {C}_{i}^{K} ,$$

can be extracted by minimizing error between calculated C_{Na} & C_{K} and their respective known concentrations of the ionic chemicals of interest in a base liquid medium applied to the sensor array 530. Said coefficients may also include an offset value, for example for i=0, C₀ is the offset value for S₀=0.

FIGS. 13C and 13D show examples of improved selectivity and accuracy for measuring concentration of potassium and sodium in a mixture of sodium chloride and potassium chloride of varying concentrations in artificial urine samples using an array of three ion-specific electrodes having different and non-zero selectivity to both K+ and Na+ ions based on above equations.

Improved accuracy and selectivity are evident when the measured ratio of the two ionic concentrations is plotted against known ratio of sodium to potassium concentration in the same solution for the three samples containing both sodium and potassium ions. As shown in FIG. 13E, potassium concentration is accurately measured even though the sodium concentration is about 7.5 times larger.

While biasing and calibration control 639 can enable charge gated transistor sensors in array 630, individual sensors are not limited to charge gated transistor sensors and other ion-specific sensors such as ion-specific electrodes can also be used as array elements, either making up the entire sensor array, or in combination with other chemical sensors.

The processing sub-system 640 is not limited to measuring two or three chemical concentrations, it can measure as many chemicals as needed, providing enough number of sensor elements are used in the sensor array.

FIG. 7 illustrates a diagram of a continues urine monitoring system used in conjunction with a catheter 720 and urine collecting bag 710. The catheter can be inserted in the patient's bladder through ureter and the other end in placed in the collecting bag. The catheter connects to the sub-system 730 that can measure urine volume using 732 and concentration of various chemicals and specific urine biomarkers such as sodium, potassium, pH, specific gravity and alike by using 734. The volume and flow sensor 732 and the chemical sensors 734 are powered and controlled by 736 which also transmit the collected data to monitoring and recording unit 740 either by wireless communication methods such as Wi-Fi, or by wire. Unit 740 can further analyze collected data, display and record data locally or on the cloud-based accounts using secure protocols via internet for further analysis or review. The continues urine monitoring system can also be used in hospitals where catheter and urine collecting bags are already in use for certain hospitalized patients.

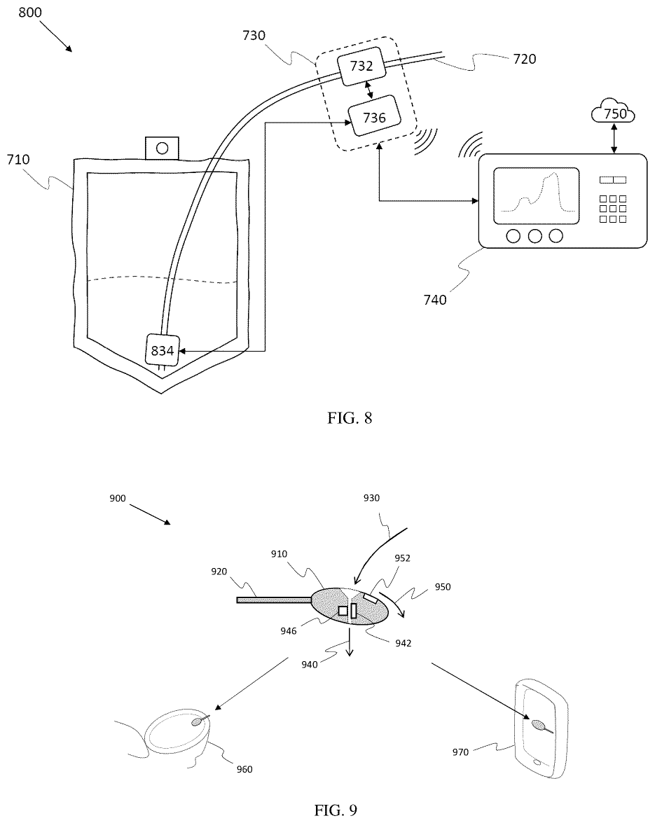

FIG. 8 illustrates a variation of the continues urine monitoring system where the chemical measurement unit 834 is placed inside the urine collecting bag, which can be connected and read any time. One advantage of system 800 is that any time the chemical sensor unit is read, measurements will represent averaged data for the entire content of the collecting bag. In system 700, chemical sensor data must be digitally integrated and or averaged over time, which may result in larger error compared to data from system 800.

FIG. 9 illustrates an example of a portable urine monitoring device 900 comprising a body 910 onto which a user can urinate, a handle 920 that a user can use to hold it while pouring urine stream 930 onto the device, an entrance for sampling urine 930 and an exit, multitude of sensors that can include chemical sensors 946 that can measure concentration of various chemicals from the portion of urine that goes into the device according to FIG. 6, sensors 942 and 952 that measure urine volume for both the portion of urine that goes into the device and finally exit as 940, and the portion of the urine 950 that flows over the device, according to FIG. 1A or FIG. 1B.

Alternatively, the handle 920 can be formed or used to mount the portable urine monitoring device 900 on a regular toilet 960, or a urinal 970.

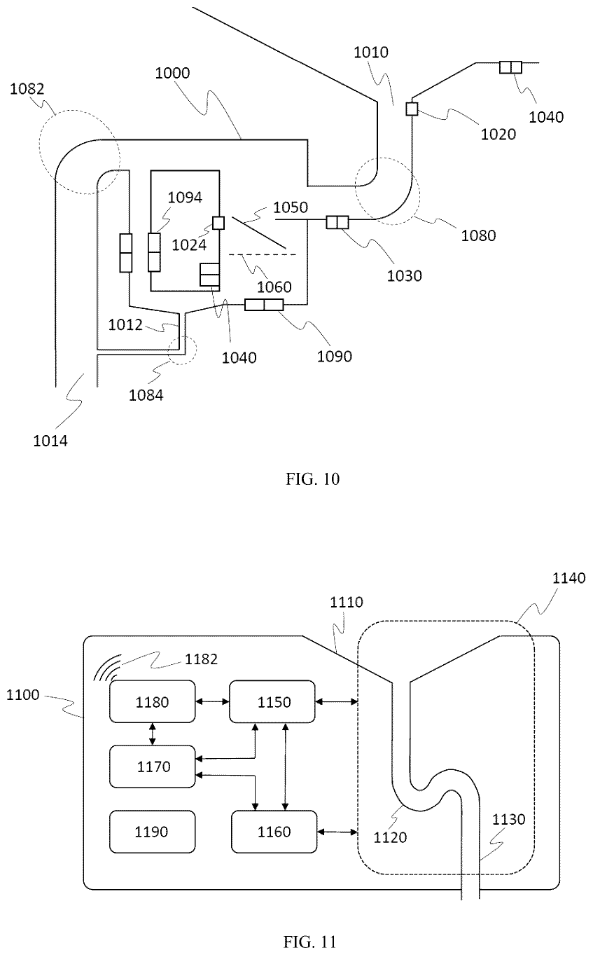

FIG. 10 illustrates details of the portable urine monitoring device shown in FIG. 9, where part of the urine may enter the body 1000 at the funnel 1010 and contacts temperature sensor 1020 and sensors 1030 down the range to measure urine temperature and urine volume, according to the system and method described in FIG. 1A or FIG. 1B, for the portion of the urine that enters 1010. For the portion of the urine that may overflow, sensors 1040 are used to measure volume according to the system and method described in FIG. 1A or FIG. 1B. Urination time and flow rate can be measured using said temperature sensors in conjunction with urine contact sensor or electrical conduction sensor 1024. A bubble trap 1050 may guides air bubbles in the urine upward, and the mesh 1060 blocks any scaping air bubbles from entering the area under 1060 such that they do not interfere with spectrometry sensors 1040, and the fold 1080 is designed to prevent ambient light from getting inside 1000 and interfering with spectroscopy sensors 1040. The quiet bubble free urine flow can flow past single or multitude of ion-specific chemical sensors such as charge gated transistor sensors 1090 or other ion-specific differential electrodes 1094 and exit via 1014. The fold 1082 may prevent ambient light entering 1000 from the exit 1014. When urine flow stops, the remaining urine inside 1000 can exit from 1012 such that no urine remains inside 1000. The fold 1084 is designed to prevent ambient light entering 1000 from 1012.

FIG. 11 illustrates a system level diagram of the portable urine monitoring device 1100 described in FIG. 9 and detailed in FIG. 10 including an entrance funnel 1010 where urine sample enters the device 1100, the U-shape pipe 1120 that conducts the urine to the exit 1130 and holds enough urine on the bottom part of the pipe for continuous measurement by multitude of sensors unit 1140 (detailed in FIG. 10) during urination.

The control unit 1150 controls operation of multitude of sensors 1140, the processing unit 1160 which processes sensors signals & data, and the communication unit 1180 which receives authentication information from user to make measurements and transfers measured data and assessment results to user's preferred device or data account wirelessly by radio waves 1182, or by wire. The power unit 1190 provides sufficient electric power to all units by harvesting energy, or from wireless energy sources, or from batteries, or from adaptors via wall electric outlets. For security, all data can be encrypted by 1170 before being transmitted by 1180. Similarly, 1180 may decrypt all communicated data received by 1180, 11 including authentication information.

FIG. 12 illustrates an example of connectivity diagram for the portable urine monitoring device 1210, where the user 1200 can activate device 1200 using their connected personal devices such as smart watch 1202 or mobile phone 1204, where the communication between the user's devices and the portable urine monitoring device 1210 can be established directly by close range communication methods such as Bluetooth or NFC, or through a common connected router/modem 1220 using Wi-Fi. Alternatively, user 1200 may activate and authenticate 1210 using a keypad 1212 which may be connected to 1210 wirelessly, or by wire. Other methods are also possible, for example activating 2010 by voice, and authenticating user 1200 using a voice recognition algorithm. Once 1210 is activated and user is authenticated, user 1200 urinated onto 1210, and collected data from user 1200 urine is transmitted to the authenticated user's cloud account 1230 via internet modem/router 1220 or directly through cellular communication, where raw data are processed by 1232 and are saved in 1234 along all other collected and processed data from user 1200. Notifications can be sent back from 1210 to said user's application on 1202 or 1204, and processed results of the very urination data can be sent back from 1230 to user's application for displaying along all or partial past data. Data can be communicated between user's cloud account and user's applications either through modem/router 1220, or directly through mobile communication services. Additionally, a physician or a care team may also be notified if any abnormality is detected in users urine data, and/or they can access the user's cloud account, if permitted, to view data and supervise the user based on results of processed data.

1.-28. (canceled)

29. An analysis system to measure urine volume during urination, comprising:
a body for receiving urine, the body is in thermal contact with a urine flow of the urine and in thermal contact with an environment; and
a temperature sensor system for thermally contacting the urine and the body, wherein the temperature sensor system is configured to measure a urine temperature and a body temperature of the body,
wherein the analysis system is configured to calculate the urine volume from a change in the body temperature over time.

30. The analysis system of claim 29, wherein the analysis system is configured to calculate the urine volume using a pure physical model, or an enhanced physical model enhanced by machine learning and/or a trainable artificial intelligent algorithm, or a pure machine learning and/or a trainable artificial intelligent algorithm model.

31. The analysis system of claim 30, wherein a parameter of the pure physical model, the enhanced physical model, and/or the pure machine learning and/or the trainable artificial intelligent algorithm model is calibrated using samples of a first volume at a first temperature.

32. The analysis system of claim 29, further comprising a processing unit, wherein the temperature sensor system is coupled to the processing unit such that a signal from the temperature sensor system is transmitted to and processed by the processing unit.

33. The analysis system of claim 32, wherein the processing unit further comprises a sampling system coupled to the temperature sensor system for sampling the signal, an edge detector coupled to the temperature sensor system and the sampling system for determining a urination start time and a urination end time, and a processor system coupled to the sampling system and the edge detector for signal and data processing.

34. The analysis system of claim 33, wherein the processor system is configured to calculate a urination time by taking a difference between the urination start time and the urination end time, wherein the urination time is calculated using a pure physical model, or an enhanced physical model enhanced by machine learning and/or a trainable artificial intelligent algorithm, or a pure machine learning and/or a trainable artificial intelligent algorithm model, wherein a parameter of the pure physical model, the enhanced physical model, and/or the pure machine learning and/or the trainable artificial intelligent algorithm model is calibrated using samples of a first duration at a first temperature.

35. The analysis system of claim 34, further comprising a liquid contact sensor coupled to the body and in fluid contact with the urine flow, wherein the liquid contact sensor is coupled to the processing unit such that a signal from the liquid contact sensor is transmitted to and processed by the processing unit, wherein the processor system is configured to calculate an enhanced urination time by incorporating the signal from the liquid contact sensor.

36. The analysis system of claim 34, wherein the processor system is configured to calculate a urine flow rate by dividing the urine volume by the urination time, wherein the processor system is further configured to evaluate a urination manner in terms of urine flow continuity and interruption.

37. The analysis system of claim 36, wherein the urine flow and/or the urination manner is calculated using a pure physical model, or an enhanced physical model enhanced by machine learning and/or a trainable artificial intelligent algorithm, or a pure machine learning and/or a trainable artificial intelligent algorithm model, wherein a parameter of the pure physical model, the enhanced physical model, and/or the pure machine learning and/or the trainable artificial intelligent algorithm model is calibrated by using samples of a first flow rate and a first flow manner at a first temperature.

38. An analysis system to measure urine volume during urination, comprising:
a body for receiving urine, the body configured to be in fluid contact with urine; and
multiple interdigitated electrodes capacitively coupled to the body, wherein a mutual capacitance among the electrodes changes as a result of the urine flowing over the body,
wherein the electrodes comprise a reference electrode fluidically separated from the urine, a drive electrode coupled to a signal generator, wherein the signal generator applies an alternating voltage to the drive electrode, and a sense electrode, wherein the sense electrode is configured to receive part of the alternating voltage via a coupling capacitance between the sense electrode and the drive electrode, and wherein the reference electrode is configured to receive part of the alternating voltage via a couple capacitance between the reference electrode and the drive electrode,
and wherein the analysis system is configured to calculate the urine volume from signals received from the sense electrode and the reference electrode using an enhanced physical model enhanced by machine learning and/or a trainable artificial intelligent algorithm, and
wherein a parameter of the enhanced physical model is calibrated using samples of a first volume.

39. The analysis system of claim 38, further comprising a processing unit, wherein the electrodes are coupled to the processing unit.

40. The analysis system of claim 39, wherein the processing unit further comprises a first processor coupled to the electrodes and a second processor in communication with the first processor.

41. The analysis system of claim 40, wherein the first processor is configured to receive a reference signal from the reference electrode and a sensed signal from the sense electrode and calculate an envelope sensed signal, the envelope sensed signal comprising the sensed signal normalized by the reference signal.

42. The analysis system of claim 41, wherein the second processor is configured to calculate a urination time by thresholding the envelope sensed signal to find a urination start time and a urination end time, and calculate a difference between the urination start time and the urination end time, wherein the first processor is further configured to integrate a ratio of the envelope sensed signal to a peak value of the reference signal over the urination time to obtain an area under the curve value.

43. The analysis system of claim 42, wherein the second processor is configured to calculate the urine volume by performing machine learning estimation from the area under the curve value.

44. The analysis system of claim 43, wherein the second processor is configured to calculate a urine flow rate from the ratio of the urine volume to the urination time.

45. An ion-specific solid-state sensor for measuring concentration of an ionic chemical in a liquid medium, comprising:
a charge gated field effect transistor comprising four electric terminals, wherein the four electric terminals comprise:
a drain,
a source,
a voltage gate, and
a charge gate,
wherein the charge gate is coated by an ion-absorbing layer, wherein the ion-absorbing layer is in contact with the liquid medium and is configured to be preferential in absorption of ions of the ionic chemical among other ionic chemicals in the liquid medium;
wherein a drain-source current of the transistor is configured to be modulated by an amount of ions absorbed by the ion-absorbing layer when a voltage bias is applied to the drain, the source, and the voltage gate, and wherein the drain-source current of the transistor is configured to be modulated by a preset charge applied to the charge gate; and
wherein an extent of a modulation of the drain-source current relates to a concentration of the ionic chemical in the liquid medium.

46. The sensor of claim 45, further comprising a gain control and an offset control, wherein the gain is adjustable by a drain-source voltage, and the offset is adjustable by a gate-source voltage and/or the preset charge applied to the charge gate when the sensor is biased in linear mode.

47. The sensor of claim 45, further comprising:
a substrate;
a buffer layer coating the substrate, wherein the drain, the source, and the voltage gate are positioned on the buffer layer;
a semiconductor layer, wherein the drain and the source are connected by the semiconductor layer; and
a dielectric layer, wherein the voltage gate and the charge gate are separated by the dielectric layer.

48. The sensor of claim 45, wherein the ion-absorbing layer comprises an ion-specific polymer membrane positioned above an inorganic ion-trapping silicon compound layer.
